(19) United States
(12) Patent Application Publication
(10) Pub. No.: US 2024/0120031 A1
(43) Pub. Date: Apr. 11, 2024

(54) COMPUTER-READABLE RECORDING MEDIUM STORING INFORMATION PROCESSING PROGRAM, INFORMATION PROCESSING METHOD, AND INFORMATION PROCESSING DEVICE

(71) Applicant: Fujitsu Limited, Kawasaki-shi (JP)

(72) Inventor: Naoki IIJIMA, Kawasaki (JP)

(73) Assignee: Fujitsu Limited, Kawasaki-shi (JP)

(21) Appl. No.: 18/323,443

(22) Filed: May 25, 2023

(30) Foreign Application Priority Data
Sep. 30, 2022 (JP) 2022-158748

(51) Int. Cl. G16C 10/00

(52) U.S. Cl. CPC G16C 10/00

(57) ABSTRACT

A recording medium stores a program for causing a computer to execute a process including: accepting selection of one or more atoms among a plurality of atoms that form a target molecule that exists in a predetermined coordinate space; and altering coordinates of each atom of the one or more atoms from first coordinates to second coordinates in the coordinate space based on: a unit vector between the coordinates of a first atom and a second atom of which a bond length is to be changed; a normal vector with respect to a plane formed by a first bond axis and a second bond axis of which a bond angle is to be changed; or a rotation matrix of the second atom of which a rotation angle is to be changed; or any combination of the unit vector, the normal vector, or the rotation matrix, in the coordinate space.

### CROSS-REFERENCE TO RELATED APPLICATION

This application is based upon and claims the benefit of priority of the prior Japanese Patent Application No. 2022-158748, filed on Sep. 30, 2022, the entire contents of which are incorporated herein by reference.

### FIELD

The embodiments discussed herein are related to an information processing program, an information processing method, and an information processing device.

### BACKGROUND

For the development of new drugs or new materials, it has been desired since earlier to generate a potential energy curve (PEC) of a target molecule. For example, it is conceivable to generate the PEC by generating a plurality of states that can be taken by the target molecule and have coordinates of atoms different from each other and calculating potential energy of the target molecule in each state.

Japanese Laid-open Patent Publication No. 2003-206246 is disclosed as related art.

### SUMMARY

According to an aspect of the embodiments, a non-transitory computer-readable recording medium stores an information processing program for causing a computer to execute a process including: accepting selection of one or more atoms among a plurality of atoms that form a target molecule that exists in a predetermined coordinate space; and altering coordinates of each atom of the one or more atoms from first coordinates to second coordinates in the coordinate space, according to designation of a change amount of: a bond length between a first atom included in the one or more atoms for which the selection has been accepted and a second atom that is not included in the one or more atoms but is coupled to the first atom; a bond angle formed by a first bond axis that bonds the first atom and the second atom and a second bond axis that bonds the second atom and a third atom that is not included in the one or more atoms but is coupled to the second atom; or a rotation angle of the second atom with respect to the second bond axis; or any combination of the bond length, the bond angle, or the rotation angle, based on: a unit vector between the coordinates of the first atom and the second atom of which the bond length is to be changed; a normal vector with respect to a plane formed by the first bond axis and the second bond axis of which the bond angle is to be changed; or a rotation matrix of the second atom of which the rotation angle is to be changed; or any combination of the unit vector, the normal vector, or the rotation matrix, in the coordinate space.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is an explanatory diagram illustrating a working example of an information processing method according to an embodiment;

FIG. 2 is an explanatory diagram illustrating an example of an information processing system 200;

FIG. 3 is a block diagram illustrating a hardware configuration example of an information processing device 100;

FIG. 4 is a block diagram illustrating a functional configuration example of the information processing device 100;

FIG. 5 is an explanatory diagram illustrating an example of acquiring coordinate data 500;

FIG. 6 is an explanatory diagram (part 1) illustrating an example of acquiring a pattern file 700;

FIG. 7 is an explanatory diagram (part 2) illustrating an example of acquiring the pattern file 700;

FIG. 8 is an explanatory diagram illustrating a first example of altering the coordinates of an atom forming a target molecule;

FIG. 9 is an explanatory diagram illustrating a second example of altering the coordinates of an atom forming a target molecule;

FIG. 10 is an explanatory diagram illustrating a third example of altering the coordinates of an atom forming a target molecule;

FIG. 11 is an explanatory diagram illustrating an example of generating a PEC;

FIG. 12 is a flowchart illustrating an example of a first acceptance processing procedure;

FIG. 13 is a flowchart illustrating an example of a second acceptance processing procedure;

FIG. 14 is a flowchart illustrating an example of a third acceptance processing procedure;

FIG. 15 is a flowchart illustrating an example of an overall processing procedure;

FIG. 16 is a flowchart illustrating an example of a first alteration processing procedure;

FIG. 17 is a flowchart illustrating an example of a second alteration processing procedure; and

FIG. 18 is a flowchart illustrating an example of a third alteration processing procedure.

### DESCRIPTION OF EMBODIMENTS

As an existing technique, for example, there is a technique in which a designated compound X is divided into a fixed rigid body unit and a moving rigid body unit, and the moving rigid body unit is translated by a specified moving distance selected at random.

Hereinafter, embodiments of an information processing program, an information processing method, and an information processing device will be described in detail with reference to the drawings.

(One Working Example of Information Processing Method According to Embodiment)

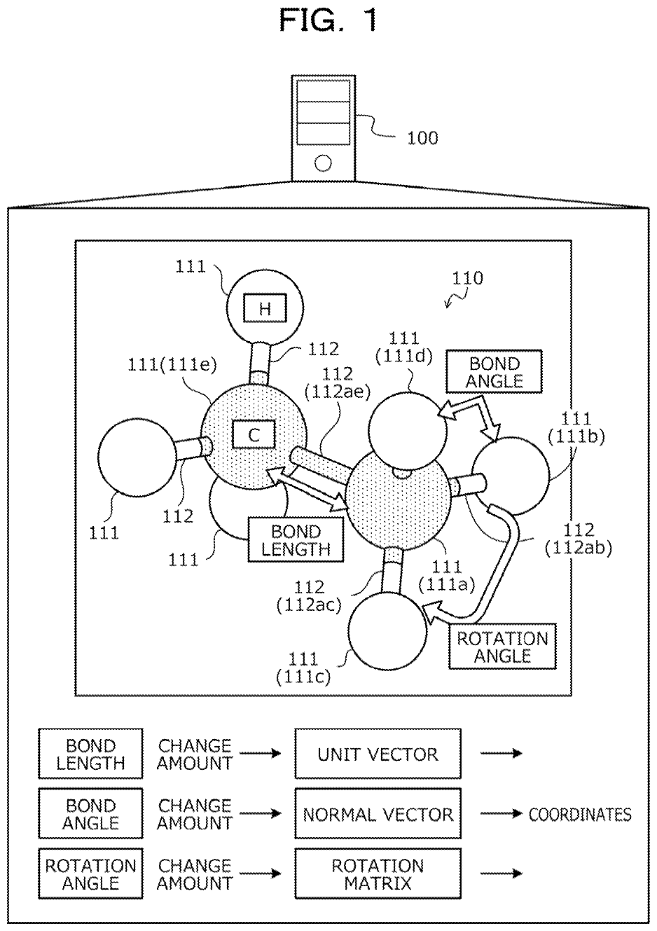

FIG. 1 is an explanatory diagram illustrating a working example of an information processing method according to an embodiment. An information processing device 100 is a computer for altering the coordinates of at least one atom of a plurality of atoms forming a target molecule existing in a predetermined coordinate space. For example, the information processing device 100 is a server, a personal computer (PC), or the like.

The coordinate space is, for example, a three-dimensional space. The target molecule is formed, for example, by bonding a plurality of atoms. For example, the target molecule is H₂O, C₂H₆, or the like. For example, the atom is H, C, or the like. FIG. 1 illustrates an example of a model 110 of a molecule. The model 110 illustrates C₂H₆, for example. The model 110 illustrates the atoms as spheres 111 and the bonds between the atoms as bars 112, for example. In the example in FIG. 1, the white spheres correspond to H. In addition, in the example in FIG. 1, the hatched spheres correspond to C.

In the following description, an axis indicating a bonding direction between atoms will be sometimes expressed as a “bond axis”. In addition, in the following description, a distance between bonded atoms will be sometimes expressed as a “bond length”. In addition, in the following description, an angle formed by bond axes from the atom at a starting point to each atom of two different atoms will be sometimes expressed as a “bond angle”. In addition, in the following description, an angle at which any one atom is rotated with a bond axis of the one atom as a rotation axis will be sometimes expressed as a “rotation angle”.

For the development of new drugs, new materials, or the like, it has been desired since earlier to generate the PEC of the target molecule and analyze the properties that the target molecule has. For example, it is conceivable to generate the PEC by generating a plurality of states that can be taken by the target molecule and have coordinates of atoms different from each other and calculating potential energy of the target molecule in each state.

For example, it is desired to change at least one of the bond length, the bond angle, or the rotation angle from a certain state of the target molecule to generate a plurality of states that can be taken by the target molecule. For example, it is desired to specify the coordinates of each atom of the plurality of atoms forming the target molecule in a new state obtained by changing at least one of the bond length, the bond angle, or the rotation angle from a certain state of the target molecule.

However, it is difficult to specify the coordinates of each atom of the plurality of atoms forming the target molecule in a new state obtained by changing at least one of the bond length, the bond angle, or the rotation angle from a certain state of the target molecule. For example, it is difficult to appropriately alter the coordinates of an atom contained in the target molecule in a certain state so as to make the coordinates correspond to a new state obtained by changing at least one of the bond length, the bond angle, or the rotation angle.

For example, when at least one of the bond length, the bond angle, or the rotation angle relating to any one atom contained in the target molecule in a certain state is changed, it is difficult to specify how to alter the coordinates of which of other atoms together with the coordinates of the one atom. Therefore, it is difficult to change at least one of the bond length, the bond angle, or the rotation angle from a certain state of the target molecule to generate a plurality of states that can be taken by the target molecule.

Thus, in the present embodiment, an information processing method capable of appropriately altering the coordinates of at least any one atom forming a target molecule existing in a predetermined coordinate space when changing at least any one of the bond length, the bond angle, or the rotation angle of the target molecule will be described.

In FIG. 1, the information processing device 100 stores the coordinates of each atom of a plurality of atoms forming a target molecule existing in a predetermined coordinate space. In the example in FIG. 1, for example, the information processing device 100 stores the coordinates of each atom of a plurality of atoms forming the molecule indicated by the model 110.

(1-1) The information processing device 100 accepts selection of one or more atoms from among a plurality of atoms forming a target molecule existing in a predetermined coordinate space. For example, the information processing device 100 accepts selection of one or more atoms whose coordinates are to be altered, from among a plurality of atoms forming a target molecule existing in a predetermined coordinate space, based on operation input from a user.

For example, the information processing device 100 accepts selection of two or more atoms indicated by spheres 111a, 111b, 111c, and 111d of the model 110 whose coordinates are to be altered according to the change in the bond length. For example, the information processing device 100 may accept selection of one atom indicated by the sphere 111b of the model 110 whose coordinates are to be altered according to the change in the bond angle. For example, the information processing device 100 may accept selection of two or more atoms indicated by the spheres 111b, 111c, and 111d of the model 110 whose coordinates are to be altered according to the change in the rotation angle.

(1-2) The information processing device 100 accepts designation of the change amount of at least one of the bond length, the bond angle, or the rotation angle relating to the one or more atoms for which selection has been accepted. For example, the information processing device 100 accepts designation of the change amount of the bond length between a first atom included in the one or more atoms for which selection has been accepted, and a second atom that is not included in the one or more atoms for which selection has been accepted but is coupled to the first atom. For example, when accepting selection of two or more atoms indicated by the spheres 111a, 111b, 111c, and 111d, the information processing device 100 accepts designation of the change amount of the bond length between the atom indicated by the sphere 111a and the atom indicated by the sphere 111e.

For example, the information processing device 100 may accept designation of the change amount of the bond angle formed by a first bond axis and a second bond axis. The first bond axis is, for example, a bond axis that bonds the first atom included in the one or more atoms for which selection has been accepted and the second atom that is not included in the one or more atoms for which selection has been accepted but is coupled to the first atom. The second bond axis is, for example, a bond axis that bonds the second atom and a third atom that is not included in the one or more atoms for which selection has been accepted but is coupled to the second atom. For example, when accepting selection of one atom indicated by the sphere 111b, the information processing device 100 may accept designation of the change amount of the bond angle formed by the bond axis indicated by the bar 112ab and the bond axis indicated by the bar 112ac.

For example, the information processing device 100 may accept designation of the change amount of the rotation angle of the second atom with respect to the second bond axis. The second atom is an atom coupled to the first atom. The second bond axis is, for example, a bond axis that bonds the second atom and the third atom that is not included in the one or more atoms for which selection has been accepted but is coupled to the second atom. For example, when accepting selection of two or more atoms indicated by the spheres 111b, 111c, and 111d, the information processing device 100 may accept designation of the change amount of the rotation angle of the atom indicated by the sphere 111a with respect to the bond axis indicated by the bar 112ae.

(1-3) The information processing device 100 alters the coordinates of each atom of the one or more atoms for which selection has been accepted, in the coordinate space, based on at least one of a unit vector, a normal vector, or a rotation matrix in the coordinate space, according to the designation of the change amount.

The unit vector is, for example, a vector having a unit length along the bond axis between the coordinates of the first atom and the second atom whose bond length is to be changed. The normal vector is, for example, a vector along a normal direction with respect to a plane formed by the first bond axis and the second bond axis whose bond angle is to be changed. The rotation matrix is, for example, a matrix that represents the rotation transformation with the coordinates of the second atom whose rotation angle is to be changed, as the origin.

For example, when accepting designation of the change amount of the bond length between the atom indicated by the sphere 111a and the atom indicated by the sphere 111e, the information processing device 100 specifies the unit vector along the bond axis indicated by the bar 112ae. For example, the information processing device 100 alters the coordinates of each atom of two or more atoms indicated by the spheres 111a, 111b, 111c, and 111d in the coordinate space, based on the specified unit vector and the change amount of the bond length.

This may allow the information processing device 100 to appropriately alter the coordinates of an atom forming the target molecule so as to make the coordinates correspond to the state obtained by changing the bond length. Therefore, the information processing device 100 may facilitate to prepare a plurality of states that can be taken by the target molecule and have different coordinates of atoms from each other and may facilitate to generate the PEC.

For example, the information processing device 100 specifies the normal vector with respect to the plane formed by the bond axis indicated by the bar 112ab and the bond axis indicated by the bar 112ac. For example, the information processing device 100 alters the coordinates of one atom indicated by the sphere 111b in the coordinate space, based on the specified normal vector and the change amount of the bond angle.

This may allow the information processing device 100 to appropriately alter the coordinates of an atom forming the target molecule so as to make the coordinates correspond to a new state obtained by changing the bond angle. Therefore, the information processing device 100 may facilitate to prepare a plurality of states that can be taken by the target molecule and have different coordinates of atoms from each other and may facilitate to generate the PEC.

For example, the information processing device 100 specifies the rotation matrix representing the rotation transformation with respect to the bond axis indicated by the bar 112ae with the coordinates of the atom indicated by the sphere 111a as the origin. For example, the information processing device 100 transforms the coordinates of each atom of two or more atoms indicated by the spheres 111b, 111c, and 111d in the coordinate space, based on the specified rotation matrix and the change amount of the rotation angle.

This may allow the information processing device 100 to appropriately alter the coordinates of an atom forming the target molecule so as to make the coordinates correspond to a new state obtained by changing the rotation angle. Therefore, the information processing device 100 may facilitate to prepare a plurality of states that can be taken by the target molecule and have different coordinates of atoms from each other and may facilitate to generate the PEC.

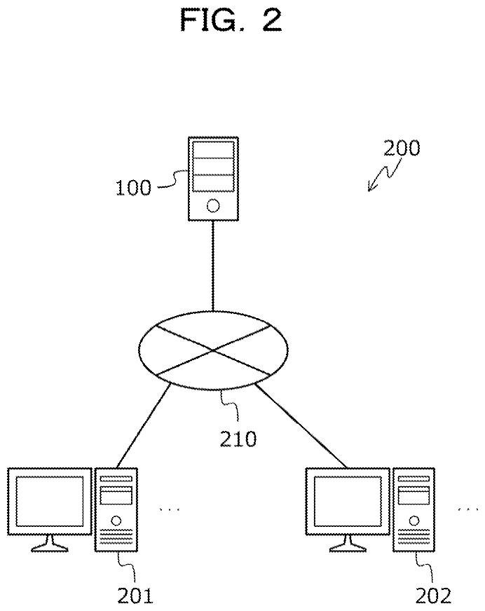

Here, the case where the information processing device 100 works independently has been described, but this is not restrictive. For example, there may be a case where the information processing device 100 cooperates with another computer. For example, there may be a case where a plurality of computers cooperates to implement a function as the information processing device 100 and works as the information processing device 100. For example, there may be a case where the function as the information processing device 100 is implemented in a cloud. An example of the case where the information processing device 100 cooperates with another computer will be described later with reference to FIG. 2.

(One Example of Information Processing System 200)

Next, an example of an information processing system 200 to which the information processing device 100 illustrated in FIG. 1 is applied will be described with reference to FIG. 2.

FIG. 2 is an explanatory diagram illustrating an example of the information processing system 200. In FIG. 2, the information processing system 200 includes the information processing device 100, an analysis device 201, and a client device 202.

In the information processing system 200, the information processing device 100 and the analysis device 201 are coupled via a wired or wireless network 210. For example, the network 210 is a local area network (LAN), a wide area network (WAN), the Internet, or the like. In addition, in the information processing system 200, the information processing device 100 and the client device 202 are coupled via the wired or wireless network 210.

The information processing device 100 is a computer for altering the coordinates of at least one atom of a plurality of atoms forming a target molecule existing in a predetermined coordinate space. The information processing device 100 receives, for example, coordinate data indicating the coordinates of each atom of a plurality of atoms forming the target molecule, from the client device 202.

For example, the information processing device 100 receives, from the client device 202, a selection notification for selecting one or more atoms whose coordinates are to be altered, from among the plurality of atoms. For example, the information processing device 100 receives, from the client device 202, a designation notification that designates the change amount of at least one of the bond length, the bond angle, or the rotation angle.

The information processing device 100 alters the coordinates of each atom of the one or more atoms for which selection has been accepted with the selection notification, in the coordinate space, according to the change amount for which designation has been accepted with the designation notification. The information processing device 100 generates new coordinate data indicating the coordinates of each atom of the plurality of atoms after the alteration.

The information processing device 100 transmits, to the analysis device 201, a request intended to calculate the potential energy of the target molecule and including the received coordinate data. The information processing device 100 receives the potential energy of the target molecule in the state indicated by the received coordinate data, from the analysis device 201.

The information processing device 100 transmits, to the analysis device 201, a request intended to calculate the potential energy of the target molecule and including the generated new coordinate data. The information processing device 100 receives the potential energy of the target molecule in the state indicated by the generated new coordinate data, from the analysis device 201.

The information processing device 100 generates the PEC, based on the received potential energy. The information processing device 100 transmits the generated PEC to the client device 202. The information processing device 100 is used by, for example, a system administrator. For example, the information processing device 100 is a server, a PC, or the like.

The analysis device 201 is a computer for calculating the potential energy of the target molecule. The analysis device 201 receives, from the information processing device 100, a request intended to calculate the potential energy of the target molecule and including the coordinate data. The analysis device 201 calculates the potential energy of the target molecule in response to the request and transmits the calculated potential energy to the information processing device 100. The analysis device 201 is used by, for example, a system administrator. For example, the analysis device 201 is a server, a PC, or the like.

The client device 202 is a computer for enabling the use of the information processing system 200. For example, the client device 202 generates the coordinate data indicating the coordinates of each atom of a plurality of atoms forming the target molecule, based on operation input from a system user, and transmits the generated coordinate data to the information processing device 100.

For example, the client device 202 generates the selection notification for selecting one or more atoms whose coordinates are to be altered, from among the plurality of atoms, based on operation input from the system user, and transmits the generated selection notification to the information processing device 100. For example, the client device 202 generates the designation notification that designates the change amount of at least one of the bond length, the bond angle, or the rotation angle, based on operation input from the system user, and transmits the generated designation notification to the information processing device 100.

The client device 202 receives, for example, the PEC from the information processing device 100. The client device 202 outputs the PEC such that the system user is allowed to refer to the PEC. The client device 202 is used, for example, by the system user. For example, the client device 202 is a PC, a tablet terminal, a smartphone, or the like.

Here, the case where the information processing device 100 and the analysis device 201 are different devices has been described, but this is not restrictive. For example, there may be a case where the information processing device 100 has a function as the analysis device 201 and works also as the analysis device 201.

Here, the case where the information processing device 100 and the client device 202 are different devices has been described, but this is not restrictive. For example, there may be a case where the information processing device 100 has a function as the client device 202 and works also as the client device 202.

(Hardware Configuration Example of Information Processing Device 100)

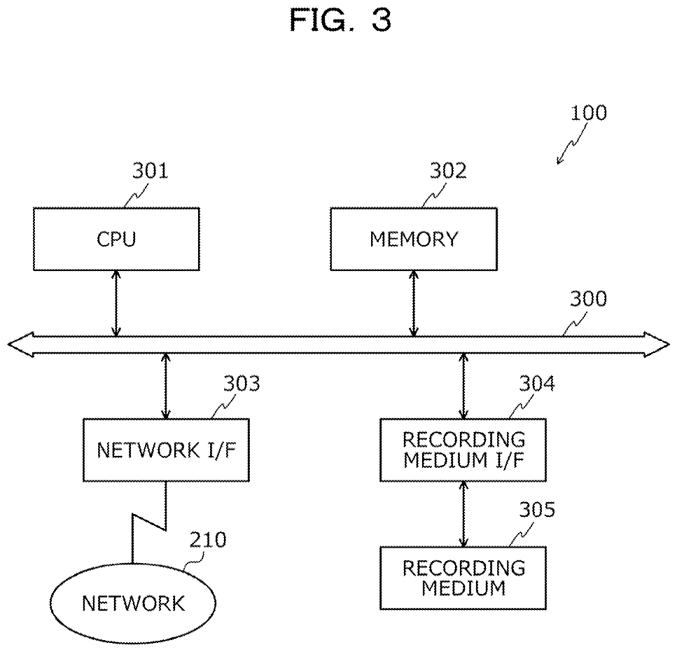

Next, a hardware configuration example of the information processing device 100 will be described with reference to FIG. 3.

FIG. 3 is a block diagram illustrating a hardware configuration example of the information processing device 100. In FIG. 3, the information processing device 100 includes a central processing unit (CPU) 301, a memory 302, a network interface (I/F) 303, a recording medium I/F 304, and a recording medium 305. In addition, the respective constituent members are inter-coupled by a bus 300.

Here, the CPU 301 takes overall control of the information processing device 100. For example, the memory 302 includes a read only memory (ROM), a random access memory (RAM), a flash ROM, and the like. For example, the flash ROM or the ROM stores various programs, and the RAM is used as a work area for the CPU 301. The programs stored in the memory 302 are loaded into the CPU 301 to cause the CPU 301 to execute coded processing.

The network I/F 303 is coupled to the network 210 through a communication line and is coupled to another computer via the network 210. Then, the network I/F 303 takes control of an interface between the network 210 and the inside and controls input and output of data from another computer. For example, the network I/F 303 is a modem, a LAN adapter, or the like.

The recording medium I/F 304 controls reading and writing of data from and to the recording medium 305 under the control of the CPU 301. For example, the recording medium I/F 304 is a disk drive, a solid state drive (SSD), a universal serial bus (USB) port, or the like. The recording medium 305 is a nonvolatile memory that stores data written under the control of the recording medium I/F 304. For example, the recording medium 305 is a disk, a semiconductor memory, a USB memory, or the like. The recording medium 305 may be attachable to and detachable from the information processing device 100.

For example, the information processing device 100 may include a keyboard, a mouse, a display, a printer, a scanner, a microphone, a speaker, or the like, as well as the constituent members described above. In addition, the information processing device 100 may include a plurality of the recording medium I/Fs 304 and the recording media 305. In addition, the information processing device 100 does not have to include the recording medium I/F 304 or the recording medium 305.

(Hardware Configuration Example of Analysis Device 201)

For example, since the hardware configuration example of the analysis device 201 is similar to the hardware configuration example of the information processing device 100 illustrated in FIG. 3, description thereof will be omitted.

(Hardware Configuration Example of Client Device 202)

For example, since the hardware configuration example of the client device 202 is similar to the hardware configuration example of the information processing device 100 illustrated in FIG. 3, description thereof will be omitted.

(Functional Configuration Example of Information Processing Device 100)

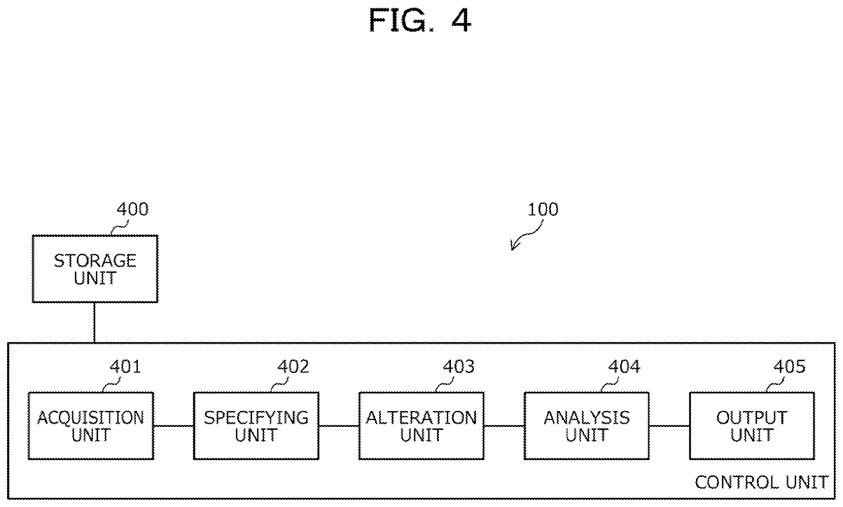

Next, a functional configuration example of the information processing device 100 will be described with reference to FIG. 4.

FIG. 4 is a block diagram illustrating a functional configuration example of the information processing device 100. The information processing device 100 includes a storage unit 400, an acquisition unit 401, a specifying unit 402, an alteration unit 403, an analysis unit 404, and an output unit 405.

The storage unit 400 is implemented by, for example, a storage area such as the memory 302 or the recording medium 305 illustrated in FIG. 3. Hereinafter, a case where the storage unit 400 is included in the information processing device 100 will be described, but this is not restrictive. For example, there may be a case where the storage unit 400 is included in a device different from the information processing device 100, and the information processing device 100 is allowed to refer to the storage contents of the storage unit 400.

The acquisition unit 401 to the output unit 405 function as an example of a control unit. The acquisition unit 401 to the output unit 405 implement functions thereof by, for example, causing the CPU 301 to execute a program stored in a storage area such as the memory 302 or the recording medium 305 or by the network I/F 303 illustrated in FIG. 3. The processing result of each functional unit is stored in, for example, a storage area such as the memory 302 or the recording medium 305 illustrated in FIG. 3.

The storage unit 400 stores various types of information to be referred to or updated in processing of each functional unit. The storage unit 400 stores the coordinates of each atom of a plurality of atoms forming a target molecule existing in a predetermined coordinate space. The coordinates of each atom are acquired by the acquisition unit 401, for example.

The acquisition unit 401 acquires various types of information to be used for processing of each functional unit. The acquisition unit 401 stores various types of the acquired information in the storage unit 400 or outputs various types of the acquired information to each functional unit. In addition, the acquisition unit 401 may output various types of information previously stored in the storage unit 400 to each functional unit. The acquisition unit 401 acquires various types of information, for example, based on operation input from a user. The acquisition unit 401 may receive various types of information from, for example, a device different from the information processing device 100.

The acquisition unit 401 acquires the coordinate data indicating the coordinates of each atom of a plurality of atoms forming the target molecule existing in a predetermined coordinate space. The acquisition unit 401 acquires the coordinate data by, for example, accepting input of the coordinate data, based on operation input from a user. The acquisition unit 401 may acquire the coordinate data by, for example, receiving the coordinate data from another computer.

For example, the acquisition unit 401 accepts selection of one or more atoms whose coordinates are to be altered, from among a plurality of atoms forming the target molecule existing in a predetermined coordinate space. For example, the acquisition unit 401 accepts selection of one or more atoms whose coordinates are to be altered, from among the plurality of atoms, based on operation input from the user. For example, the acquisition unit 401 may accept selection of one or more atoms by receiving, from another computer, the selection notification for selecting one or more atoms whose coordinates are to be altered, from among the plurality of atoms.

For example, the acquisition unit 401 accepts designation of the change amount of the bond length between the first atom and the second atom. The unit of the change amount is, for example, a unit of length. For example, the unit of the change amount is nanometer, angstrom, or the like. The first atom is, for example, an atom whose coordinates are to be altered, among the plurality of atoms. For example, the first atom corresponds to an atom included in the one or more atoms for which selection has been accepted. The second atom is, for example, an atom whose coordinates are not to be altered, among the plurality of atoms, and is an atom coupled to the first atom. For example, the second atom corresponds to an atom that is not included in the one or more atoms for which selection has been accepted. For example, the acquisition unit 401 accepts designation of the change amount of the bond length, based on operation input from the user. For example, the acquisition unit 401 may accept designation of the change amount of the bond length by receiving the designation notification that designates the change amount of the bond length, from another computer.

For example, the acquisition unit 401 accepts designation of the change amount of the bond angle formed by the first bond axis between the first atom and the second atom and the second bond axis between the second atom and the third atom. The bond axis bonds two different atoms. The unit of the change amount is, for example, an angle. The first atom is, for example, an atom whose coordinates are to be altered, among the plurality of atoms. For example, the first atom corresponds to an atom included in the one or more atoms for which selection has been accepted. The second atom is, for example, an atom whose coordinates are not to be altered, among the plurality of atoms, and is an atom coupled to the first atom. For example, the second atom corresponds to an atom that is not included in the one or more atoms for which selection has been accepted. The third atom is, for example, an atom whose coordinates are not to be altered, among the plurality of atoms, and is an atom coupled to the second atom. For example, the third atom corresponds to an atom that is not included in the one or more atoms for which selection has been accepted. For example, the acquisition unit 401 accepts designation of the change amount of the bond angle, based on operation input from the user. For example, the acquisition unit 401 may accept designation of the change amount of the bond angle by receiving the designation notification that designates the change amount of the bond angle, from another computer.

For example, the acquisition unit 401 accepts designation of the change amount of the rotation angle of the second atom with respect to the second bond axis between the second atom and the third atom. The unit of the change amount is, for example, an angle. The second atom is, for example, an atom whose coordinates are not to be altered, among the plurality of atoms, and is an atom coupled to the first atom. For example, the second atom corresponds to an atom that is not included in the one or more atoms for which selection has been accepted. The third atom is, for example, an atom whose coordinates are not to be altered, among the plurality of atoms, and is an atom coupled to the second atom. For example, the third atom corresponds to an atom that is not included in the one or more atoms for which selection has been accepted. For example, the acquisition unit 401 accepts designation of the change amount of the rotation angle, based on operation input from the user. For example, the acquisition unit 401 may accept designation of the change amount of the rotation angle by receiving the designation notification that designates the change amount of the rotation angle, from another computer.

For example, the acquisition unit 401 may accept selection of one or more atoms whose coordinates are to be altered, from among the plurality of atoms, and may also accept designation of the change amount of at least one of the bond length, the bond angle, or the rotation angle. For example, after accepting selection of one or more atoms whose coordinates are to be altered, from among the plurality of atoms, the acquisition unit 401 may accept designation of the change amount of at least one of the bond length, the bond angle, or the rotation angle. For example, before accepting selection of one or more atoms whose coordinates are to be altered, from among the plurality of atoms, the acquisition unit 401 may accept designation of the change amount of at least one of the bond length, the bond angle, or the rotation angle.

For example, the acquisition unit 401 may accept selection of the first atom whose coordinates are to be altered, from among a plurality of atoms forming the target molecule existing in a predetermined coordinate space. For example, the acquisition unit 401 may accept selection of the first atom whose coordinates are to be altered, from among the plurality of atoms, based on operation input from the user. For example, the acquisition unit 401 may accept selection of the first atom by receiving, from another computer, the selection notification for selecting the first atom whose coordinates are to be altered, from among the plurality of atoms.

For example, the acquisition unit 401 may accept selection of another atom linked with the first atom, by selecting the another atom from among the plurality of atoms. For example, the acquisition unit 401 accepts selection of another atom linked with the first atom, by automatically selecting the another atom when the bond length, the bond angle, and the rotation angle are changed according to designation of the change amount.

The acquisition unit 401 may accept a start trigger to start processing of any functional unit. The start trigger is, for example, a predetermined operation input made by the user. The start trigger may be, for example, reception of predetermined information from another computer. The start trigger may be, for example, output of predetermined information by any functional unit. For example, the acquisition unit 401 may accept the acceptance of selection of the one or more atoms and the acceptance of designation of the change amount, as a start trigger for starting processing of the specifying unit 402 and the alteration unit 403.

The specifying unit 402 specifies the unit vector between the coordinates of the first atom and the second atom whose bond length is to be changed, in the coordinate space, according to designation of a first change amount of the bond length between the first atom and the second atom. The unit vector is, for example, a unit vector between the coordinates of the first atom and the second atom in a state before the bond length is actually changed. The unit vector is, for example, a vector having a unit length. Note that the unit vector is a vector corresponding to the bond axis that bonds the first atom and the second atom. For example, among the first atom and the second atom whose bond length is to be changed, the specifying unit 402 specifies the unit vector from the coordinates of the second atom whose coordinates are not to be altered, to the coordinates of the first atom whose coordinates are to be altered. This allows the specifying unit 402 to obtain information that allows the coordinates of each atom of the one or more atoms for which selection has been accepted, to be altered according to the change in the bond length.

The specifying unit 402 specifies the normal vector with respect to the plane formed by the first bond axis and the second bond axis whose bond angle is to be changed, in the coordinate space, according to designation of a second change amount of the bond angle formed by the first bond axis and the second bond axis. The normal vector is, for example, a normal vector with respect to a plane formed by the first bond axis and the second bond axis in a state before the bond angle is actually changed. For example, the specifying unit 402 calculates, as the normal vector, an outer product of a vector along the first bond axis from the coordinates of the second atom to the coordinates of the first atom and a vector along the second bond axis from the coordinates of the second atom to the coordinates of the third atom. This allows the specifying unit 402 to obtain information that allows the coordinates of each atom of the one or more atoms for which selection has been accepted, to be altered according to the change in the bond angle.

The specifying unit 402 specifies the rotation matrix of the second atom whose rotation angle is to be changed, in the coordinate space, according to designation of a third change amount of the rotation angle of the second atom with respect to the second bond axis. For example, the specifying unit 402 specifies a rotation matrix representing the rotation transformation with the coordinates of the second atom as the origin, using unit vectors in three directions that are horizontal or vertical to the second bond axis assumed as the rotation axis and are vertical to each other. This allows the specifying unit 402 to obtain information that allows the coordinates of each atom of the one or more atoms for which selection has been accepted, to be altered according to the change in the rotation angle.

The alteration unit 403 alters the coordinates of each atom of the one or more atoms for which selection has been accepted. For example, the alteration unit 403 alters the coordinates of each atom of the one or more atoms for which selection has been accepted, from first coordinates to second coordinates. The first coordinates correspond to, for example, the coordinates before the alteration. The second coordinates correspond to, for example, coordinates after the alteration.

For example, the alteration unit 403 alters the coordinates of each atom of the one or more atoms in the coordinate space, based on the specified unit vector, according to designation of the first change amount of the bond length between the first atom and the second atom. For example, the alteration unit 403 alters the first coordinates of each atom of the one or more atoms to the second coordinates at the destination after being moved from the first coordinates by an amount equal to the unit vector multiplied by the first change amount. This may allow the alteration unit 403 to appropriately specify the coordinates of each atom of the plurality of atoms forming the target molecule in a state in which the bond length has been changed and to appropriately generate a state in which the bond length has been changed.

For example, the alteration unit 403 alters the coordinates of each atom of the one or more atoms in the coordinate space, based on the specified normal vector, according to designation of the second change amount of the bond angle formed by the first bond axis and the second bond axis. For example, based on the second change amount, the vector from the coordinates of the second atom to the coordinates of the third atom, and the normal vector, the alteration unit 403 specifies a first vector from the coordinates of the second atom to the first coordinates of the first atom after the change in the bond angle. For example, the alteration unit 403 alters the first coordinates of the first atom to the second coordinates at the destination after being moved from the first coordinates by an amount equal to the specified first vector.

For example, if the one or more atoms include an atom other than the first atom, the alteration unit 403 specifies a second vector from the first coordinates to the second coordinates of the first atom before and after the change in the bond angle. For example, the alteration unit 403 alters the first coordinates of each atom other than the first atom among the one or more atoms to the second coordinates at the destination after being moved from the first coordinates by an amount equal to the specified second vector. This may allow the alteration unit 403 to appropriately specify the coordinates of each atom of the plurality of atoms forming the target molecule in a state in which the bond angle has been changed and to appropriately generate a state in which the bond angle has been changed.

For example, the alteration unit 403 alters the coordinates of each atom of the one or more atoms in the coordinate space, based on the specified rotation matrix, according to designation of the third change amount of the rotation angle of the second atom with respect to the second bond axis. For example, the alteration unit 403 alters the first coordinates of the first atom to the second coordinates obtained by multiplying the first coordinates by the rotation matrix corresponding to the third change amount.

For example, if the one or more atoms include an atom other than the first atom, the alteration unit 403 specifies a third vector from the first coordinates to the second coordinates of the first atom before and after the change in the rotation angle. For example, the alteration unit 403 alters the first coordinates of each atom other than the first atom among the one or more atoms to the second coordinates at the destination after being moved from the first coordinates by an amount equal to the specified third vector. This may allow the alteration unit 403 to appropriately specify the coordinates of each atom of the plurality of atoms forming the target molecule in a state in which the rotation angle has been changed and to appropriately generate a state in which the rotation angle has been changed.

For example, the alteration unit 403 may alter the coordinates of each atom of the one or more atoms in the coordinate space, based on at least one of the unit vector, the normal vector, or the rotation matrix in the coordinate space, according to the change amount for each designation of the change amount. This allows the alteration unit 403 to specify a plurality of states of the target molecule in which at least one of the bond length, the bond angle, or the rotation angle is changed.

The analysis unit 404 calculates the potential energy of the target molecule in a case where the coordinates of each atom of the one or more atoms in the coordinate space are altered according to the change amount for each designation of the change amount. The analysis unit 404 generates the PEC of the target molecule, based on the calculated potential energy. This allows the analysis unit 404 to generate the PEC and to make it easy for the user to analyze the properties of the target molecule.

The output unit 405 outputs a processing result of at least any one of the functional units. The output format is, for example, display on a display, print output to a printer, transmission to an external device by the network I/F 303, or storage in a storage area such as the memory 302 or the recording medium 305. This may allow the output unit 405 to make it possible for the user to be notified of the processing result of at least any one of the functional units, to support the management and service of the information processing device 100, such as update of the setting values of the information processing device 100 as an example, and to improve the convenience of the information processing device 100.

The output unit 405 outputs, for example, the coordinate data indicating the coordinates of each atom of the plurality of atoms forming the target molecule, including the coordinates of each atom of the one or more atoms altered by the alteration unit 403. For example, the output unit 405 outputs the coordinate data such that the user is allowed to refer to the coordinate data. This allows the output unit 405 to make it possible for the user to utilize a variety of types of the coordinate data.

The output unit 405 may output, for example, the PEC generated by the analysis unit 404. For example, the output unit 405 outputs the PEC such that the user is allowed to refer to the PEC. This allows the output unit 405 to make it possible for the user to refer to the PEC and to easily analyze the properties of the target molecule.

(Example of Working of Information Processing Device 100)

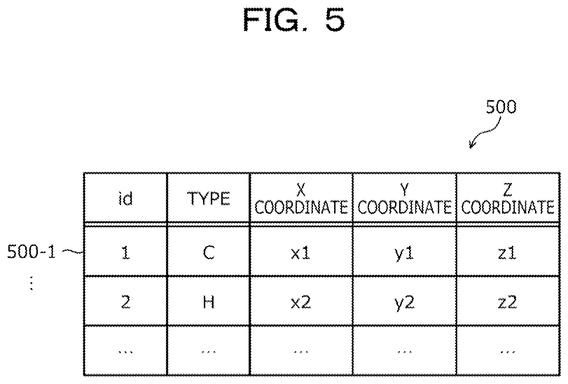

Next, an example of working of the information processing device 100 will be described with reference to FIGS. 5 to 11. First, an example in which the information processing device 100 acquires coordinate data 500 indicating the coordinates of each atom of a plurality of atoms forming a target molecule will be described with reference to FIG. 5.

FIG. 5 is an explanatory diagram illustrating an example of acquiring the coordinate data 500. In FIG. 5, the information processing device 100 acquires the coordinate data 500. As illustrated in FIG. 5, the coordinate data 500 has fields for an id, a type, an X coordinate, a Y coordinate, and a Z coordinate. In the coordinate data 500, coordinate information is stored as a record 500-a by setting information in each field for each atom. Any integer is denoted by a.

In the id field, the id that identifies an atom forming the target molecule is set. In the type field, the atomic symbol indicating the type of the above atom is set. In the X coordinate field, the X coordinate of the above atom in the coordinate space of the X, Y, and Z axes is set. In the Y coordinate field, the Y coordinate of the above atom in the coordinate space of the X, Y, and Z axes is set. In the Z coordinate field, the Z coordinate of the above atom in the coordinate space of the X, Y, and Z axes is set.

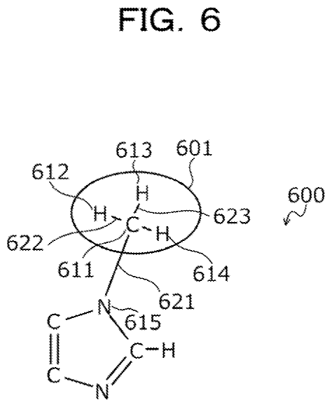

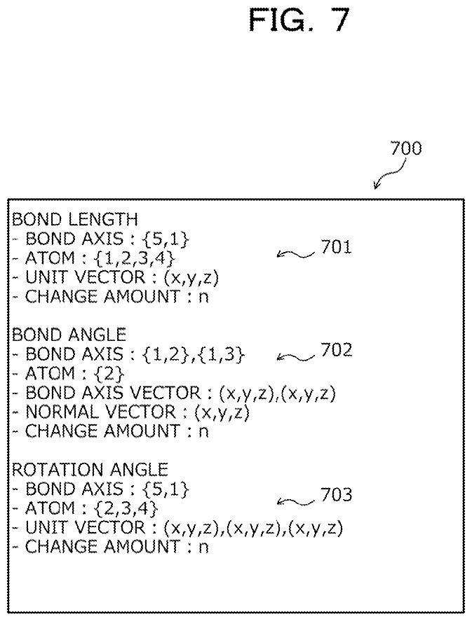

Next, an example in which the information processing device 100 acquires a pattern file 700 recording one or more alteration patterns each indicating how to alter at least one of the bond length, the bond angle, or the rotation angle will be described with reference to FIGS. 6 and 7.

FIGS. 6 and 7 are explanatory diagrams illustrating an example of acquiring the pattern file 700. In FIG. 6, the information processing device 100 displays a structural formula 600 of the target molecule on a display such that the user is allowed to refer to the structural formula 600. The information processing device 100 accepts designation of the type as a target to be changed among the bond length, the bond angle, and the rotation angle relating to the target molecule, based on operation input from the user.

For example, it is a conceivable case that the information processing device 100 accepts designation of the bond length relating to the target molecule as the type as the target to be changed. In this case, for example, the information processing device 100 accepts selection of the bond axis whose bond length is to be changed, based on operation input from the user. For example, the information processing device 100 accepts selection of a bond axis 621 between an atom 611 of C and an atom 615 of N.

For example, the information processing device 100 accepts selection of one or more atoms whose coordinates are to be altered according to the change in the bond length, from among a plurality of atoms forming the target molecule, based on operation input from the user. For example, the information processing device 100 accepts selection of a group 601 of atoms 611 to 614 whose coordinates are to be altered according to the change in the bond length, from the structural formula 600 of the target molecule, based on operation input from the user.

For example, among two atoms coupled to the bond axis for which selection has been accepted, the information processing device 100 specifies the unit vector along the direction from the coordinates of one atom whose coordinates are to be altered, to another atom whose coordinates are not to be altered. For example, the information processing device 100 specifies the unit vector along the direction from the coordinates of the atom 611 of C to the coordinates of the atom 615 of N. For example, the information processing device 100 accepts designation of the change amount of the bond length, based on operation input from the user. Next, description of FIG. 7 will be given.

In FIG. 7, the information processing device 100 generates an alteration pattern 701 in which the type as the target to be changed=the bond length, the bond axis and the one or more atoms for which selection has been accepted, the specified unit vector, and the change amount of the bond length for which designation has been accepted are associated with each other. The information processing device 100 records the generated alteration pattern 701 in the pattern file 700.

The alteration pattern 701 indicates the bond axis by including, for example, the combination of the ids of the respective atoms of two atoms coupled to the bond axis. The alteration pattern 701 indicates the one or more atoms by including, for example, the id of each atom of the one or more atoms. The alteration pattern 701 indicates the unit vector by including, for example, an x component, a y component, and a z component of the unit vector. The alteration pattern 701 includes, for example, the change amount of the bond length.

The information processing device 100 may record a plurality of alteration patterns 701 corresponding to cases of changing the bond length, in the pattern file 700. This allows the information processing device 100 to make it possible to specify how to change the bond length relating to the target molecule and how to alter the coordinates of an atom forming the target molecule.

In addition, for example, it is a conceivable case that the information processing device 100 accepts designation of the bond angle relating to the target molecule as the type as the target to be changed. In this case, for example, the information processing device 100 accepts selection of the combination of two bond axes whose bond angle is to be changed, based on operation input from the user. For example, the information processing device 100 accepts selection of the combination of a bond axis 622 between the atom 611 of C and the atom 612 of H and a bond axis 623 between the atom 611 of C and the atom 613 of H.

For example, the information processing device 100 accepts selection of one or more atoms whose coordinates are to be altered according to the change in the bond angle, from among the plurality of atoms forming the target molecule, based on operation input from the user. For example, the information processing device 100 accepts selection of the atom 612 whose coordinates are to be altered according to the change in the bond angle, from the structural formula 600 of the target molecule, based on operation input from the user.

For example, the information processing device 100 specifies bond axis vectors from the coordinates of the atom at the center of the two bond axes of the combination for which selection has been accepted, to the coordinates of the other atom of each bond axis. The atom at the center of the two bond axes is, for example, an atom commonly coupled to the two bond axes. For example, the information processing device 100 specifies the bond axis vector along the direction from the coordinates of the atom 611 of C to the coordinates of the atom 612 of H and the bond axis vector along the direction from the coordinates of the atom 611 of C to the coordinates of the atom 613 of H.

For example, the information processing device 100 specifies the normal vector with respect to the plane formed by the two bond axes of the combination for which selection has been accepted. For example, the information processing device 100 specifies the normal vector with respect to the plane formed by the bond axis 622 between the atom 611 of C and the atom 612 of H and the bond axis 623 between the atom 611 of C and the atom 613 of H. For example, the information processing device 100 accepts designation of the change amount of the bond angle, based on operation input from the user.

The information processing device 100 generates an alteration pattern 702 in which the type as the target to be changed=the bond angle, the combination and the one or more atoms for which selection has been accepted, the specified two bond axis vectors and normal vector, and the change amount of the bond angle for which designation has been accepted are associated with each other. The information processing device 100 records the generated alteration pattern 702 in the pattern file 700.

The alteration pattern 702 indicates the combination of two bond axes by including, for example, the combinations of the ids of the respective atoms of two atoms coupled to the respective bond axes of the two bond axes. The alteration pattern 702 indicates the one or more atoms by including, for example, the id of each atom of the one or more atoms. The alteration pattern 702 indicates the bond axis vectors by including, for example, the x components, the y components, and the z components of the bond axis vectors. The alteration pattern 702 indicates the normal vector by including, for example, the x component, the y component, and the z component of the normal vector. The alteration pattern 702 includes, for example, the change amount of the bond angle.

The information processing device 100 may record a plurality of alteration patterns 702 in the pattern file 700. This allows the information processing device 100 to make it possible to specify how to change the bond angle relating to the target molecule and how to alter the coordinates of an atom forming the target molecule.

In addition, for example, it is a conceivable case that the information processing device 100 accepts designation of the rotation angle relating to the target molecule as the type as the target to be changed. In this case, for example, the information processing device 100 accepts selection of the bond axis to be assumed as the rotation axis, based on operation input from the user. For example, the information processing device 100 accepts selection of a bond axis 621 between the atom 611 of C and the atom 615 of N.

For example, the information processing device 100 accepts selection of one or more atoms whose coordinates are to be altered according to the change in the rotation angle, from among the plurality of atoms forming the target molecule, based on operation input from the user. For example, the information processing device 100 accepts selection of the atoms 612 to 614 whose coordinates are to be altered according to the change in the rotation angle, from the structural formula 600 of the target molecule, based on operation input from the user.

For example, the information processing device 100 specifies unit vectors in three directions that are each horizontal or vertical to the bond axis for which selection has been accepted and are vertical to each other. For example, the information processing device 100 specifies the unit vectors in three directions that are each horizontal or vertical to the bond axis 621 and are vertical to each other. For example, the information processing device 100 accepts designation of the change amount of the rotation angle, based on operation input from the user.

The information processing device 100 generates an alteration pattern 703 in which the type as the target to be changed=the rotation angle, the bond axis for which selection has been accepted, the specified unit vectors in the three directions, and the change amount of the rotation angle for which designation has been accepted are associated with each other. The information processing device 100 records the generated alteration pattern 703 in the pattern file 700.

The alteration pattern 703 indicates the bond axis to be assumed as the rotation axis by including, for example, the combination of the ids of the respective atoms of two atoms coupled to the bond axis assumed as the rotation axis. The alteration pattern 703 indicates the one or more atoms by including, for example, the id of each atom of the one or more atoms. The alteration pattern 703 indicates the unit vectors by including, for example, the x components, the y components, and the z components of the unit vectors. The alteration pattern 703 includes, for example, the change amount of the rotation angle.

The information processing device 100 may record a plurality of alteration patterns 703 in the pattern file 700. This allows the information processing device 100 to make it possible to specify how to change the rotation angle relating to the target molecule and how to alter the coordinates of an atom forming the target molecule.

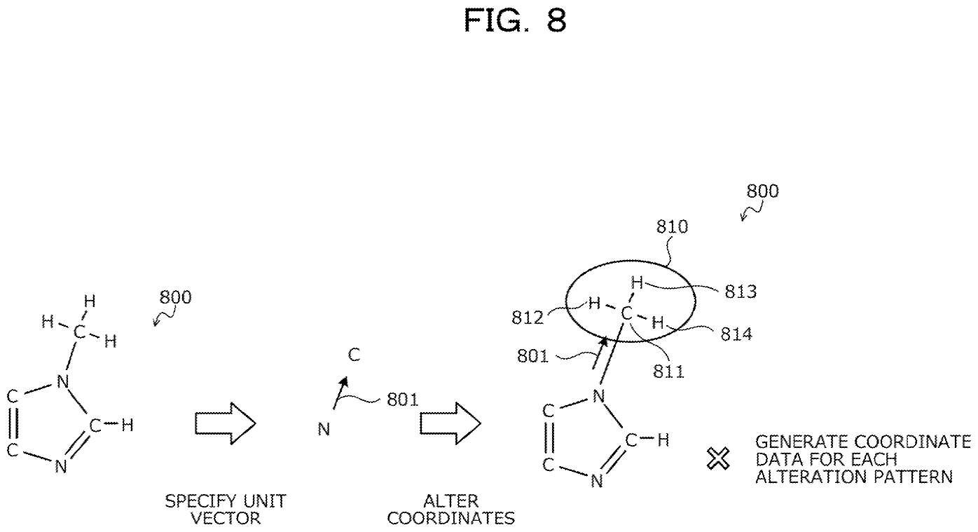

Next, a first example in which the information processing device 100 alters the coordinates of an atom forming the target molecule so as to make the coordinates correspond to the state obtained by changing the bond length, based on the alteration pattern 701 regarding the bond length, will be described with reference to FIG. 8.

FIG. 8 is an explanatory diagram illustrating the first example of altering the coordinates of an atom forming a target molecule. In FIG. 8, the target molecule is assumed as, for example, a molecule 800. For example, the target molecule is methylimidazole or the like.

Here, as in FIGS. 6 and 7, it is a conceivable case that the information processing device 100 accepts selection of atoms 811 to 814 whose coordinates are to be altered for the molecule 800 and records a plurality of alteration patterns 701 regarding the bond length in the pattern file 700. In this case, an example in which the information processing device 100 alters the coordinates of an atom forming the target molecule, based on the alteration patterns 701, will be described.

The information processing device 100 refers to the pattern file 700 to extract any alteration pattern 701 regarding the bond length. The information processing device 100 specifies a unit vector 801 of the target molecule 800, based on the extracted alteration pattern 701.

The information processing device 100 specifies the atoms 811 to 814 whose coordinates are to be altered, based on the extracted alteration pattern 701. The information processing device 100 alters the coordinates of each of the specified atoms 811 to 814 to the coordinates at the destination after being moved from the coordinates by an amount equal to the unit vector 801 multiplied by the change amount of the bond length. The information processing device 100 generates the coordinate data indicating the coordinates of each atom of a plurality of atoms forming the target molecule, including the altered coordinates.

In this manner, the information processing device 100 alters the coordinates of an atom forming the target molecule for each alteration pattern 701 regarding the bond length and generates the coordinate data. This allows the information processing device 100 to specify a plurality of different states of the target molecule regarding the bond length. For example, the information processing device 100 may appropriately specify the coordinates of each atom of a plurality of atoms forming the target molecule in a plurality of different states of the target molecule.

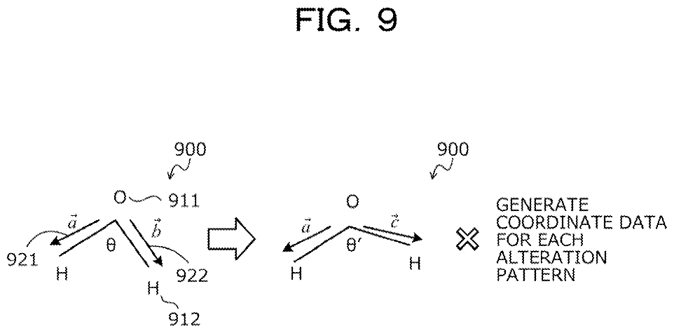

Next, a second example in which the information processing device 100 alters the coordinates of an atom forming the target molecule so as to make the coordinates correspond to the state obtained by changing the bond angle, based on the alteration pattern 702 regarding the bond angle, will be described with reference to FIG. 9.

FIG. 9 is an explanatory diagram illustrating the second example of altering the coordinates of an atom forming the target molecule. In FIG. 9, the target molecule is assumed as, for example, a molecule 900. For example, the target molecule is H₂O or the like.

Here, as in FIGS. 6 and 7, it is a conceivable case that the information processing device 100 accepts selection of an atom 911 whose coordinates are to be altered for the molecule 900 and records a plurality of alteration patterns 702 regarding the bond angle in the pattern file 700. In this case, an example in which the information processing device 100 alters the coordinates of an atom forming the target molecule, based on the alteration patterns 702, will be described.

The information processing device 100 refers to the pattern file 700 to extract any alteration pattern 702 regarding the bond angle. The information processing device 100 specifies a bond axis vector 921 and a bond axis vector 922 of the target molecule 900, based on the extracted alteration pattern 702. The information processing device 100 specifies an atom 912 whose coordinates are to be altered, based on the extracted alteration pattern 702. The information processing device 100 specifies a bond angle θ′ after the change, based on the change amount of the bond angle, based on the extracted alteration pattern 702.

In the following description, for convenience, a symbol with → attached to an upper portion will be sometimes expressed as a “symbol→”. The bond axis vector 921 is denoted by a→. The bond axis vector 922 is denoted by b→. The information processing device 100 specifies the normal vector of the target molecule 900, based on the extracted alteration pattern 702. The normal vector is denoted by a→×b→. An outer product is indicated by ×. The information processing device 100 sets the bond axis vector 922 after changing the bond angle as c→. Here, regarding a→, b→, and c→, following formula (1), following formula (2), and following formula (3) hold.

$$\begin{matrix}[ Mathematical Formula 1 ] & \\ cos {θ}^{′} = \frac{\vec{a} \cdot \vec{c}}{\vert \vec{a} \vert \vert \vec{c} \vert} & ( 1 )\end{matrix}$$

$$\begin{matrix}[ Mathematical Formula 2 ] & \\ ( \vec{a} \times \vec{b} ) \cdot \vec{c} = 0 & ( 2 )\end{matrix}$$

$$\begin{matrix}[ Mathematical Formula 3 ] & \\ \vert \vec{b} \vert = \vert \vec{c} \vert & ( 3 )\end{matrix}$$

Accordingly, based on the specified θ′, the information processing device 100 specifies c→representing the bond axis vector 922 after the change such that above formula (1), above formula (2), and above formula (3) hold. The information processing device 100 alters the coordinates of the atom 911 to the coordinates at the destination after being moved from the coordinates of the atom 912 by an amount equal to c→.

If there is another atom linked with the atom 911, the information processing device 100 alters the coordinates of the another atom. For example, when there is another atom linked with the atom 911, the information processing device 100 specifies a vector (c→−b→) between the coordinates of the atom 911 before and after the change. For example, the information processing device 100 alters the coordinates of the another atom to the coordinates at the destination after being moved from the coordinates by an amount equal to the vector (c→−b→). The information processing device 100 generates the coordinate data indicating the coordinates of each atom of a plurality of atoms forming the target molecule, including the altered coordinates.

In this manner, the information processing device 100 alters the coordinates of an atom forming the target molecule for each alteration pattern 702 regarding the bond angle and generates the coordinate data. This allows the information processing device 100 to specify a plurality of different states of the target molecule regarding the bond angle. For example, the information processing device 100 may appropriately specify the coordinates of each atom of a plurality of atoms forming the target molecule in a plurality of different states of the target molecule.

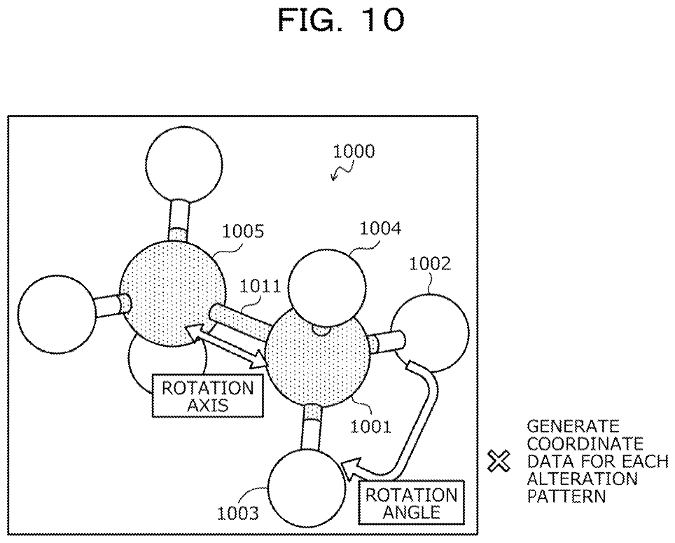

Next, a third example in which the information processing device 100 alters the coordinates of an atom forming the target molecule so as to make the coordinates correspond to the state obtained by changing the rotation angle, based on the alteration pattern 703 regarding the rotation angle, will be described with reference to FIG. 10.

FIG. 10 is an explanatory diagram illustrating the third example of altering the coordinates of an atom forming the target molecule. In FIG. 10, the target molecule is assumed as, for example, a molecule 1000. For example, the target molecule is ethane or the like.

Here, as in FIGS. 6 and 7, it is a conceivable case that the information processing device 100 accepts selection of atoms 1002 to 1004 whose coordinates are to be altered for the molecule 1000 and records a plurality of alteration patterns 703 regarding the rotation angle in the pattern file 700. In this case, an example in which the information processing device 100 alters the coordinates of an atom forming the target molecule, based on the alteration patterns 703, will be described.

The information processing device 100 refers to the pattern file 700 to extract any alteration pattern 703 regarding the rotation angle. The information processing device 100 specifies a bond axis vector 1011 of the target molecule 1000, which is assumed as the rotation axis, based on the extracted alteration pattern 703. The atom 1001 is a target to be rotated about the rotation axis. The information processing device 100 specifies the atoms 1002 to 1004 whose coordinates are to be altered, based on the extracted alteration pattern 703.

The information processing device 100 specifies unit vectors n₁, n₂, and n₃ in the three directions, based on the extracted alteration pattern 703. The information processing device 100 specifies a rotation matrix R(θ) indicated by following formula (4), based on a change amount θ of the rotation angle and the unit vectors n₁, n₂, and n₃ in the three directions.

$$\begin{matrix}[ Mathematical Formula 4 ] & \\ R ( θ ) = ( \begin{matrix}{n}_{1}^{2} ( 1 - cos θ ) + cos θ & {n}_{1} {n}_{2} ( 1 - cos θ ) - {n}_{3} sin θ & {n}_{1} {n}_{3} ( 1 - cos θ ) - {n}_{2} sin θ \\ {n}_{1} {n}_{2} ( 1 - cos θ ) + {n}_{3} sin θ & {n}_{2}^{2} ( 1 - cos θ ) + cos θ & {n}_{2} {n}_{3} ( 1 - cos θ ) - {n}_{1} sin θ \\ {n}_{1} {n}_{3} ( 1 - cos θ ) - {n}_{2} sin θ & {n}_{2} {n}_{3} ( 1 - cos θ ) + {n}_{1} sin θ & {n}_{3}^{2} ( 1 - cos θ ) + cos θ\end{matrix} ) & ( 4 )\end{matrix}$$

The information processing device 100 alters the coordinates of each of the atoms 1002 to 1004 directly coupled to the atom 1001 to coordinates obtained by multiplying the coordinates by the rotation matrix R(θ). If there is another atom linked with any atom of the respective atoms 1002 to 1004 directly coupled to the atom 1001, the information processing device 100 alters the coordinates of the another atom.

For example, if there is another atom linked with any atom of the respective atoms 1002 to 1004, the information processing device 100 specifies a vector between the coordinates of each of the atoms 1002 to 1004 before and after the alteration. For example, the information processing device 100 alters the coordinates of the another atom linked with any atom of the respective atoms 1002 to 1004 to the coordinates at the destination after being moved from the coordinates by an amount equal to the vector between the coordinates of any atom of the respective atoms 1002 to 1004 before and after the alteration. The information processing device 100 generates the coordinate data indicating the coordinates of each atom of a plurality of atoms forming the target molecule, including the altered coordinates. For example, the information processing device 100 alters the coordinates of another atom linked with the atom 1002 to the coordinates at the destination after being moved from the coordinates by an amount equal to the vector between the coordinates of the atom 1002 before and after the alteration.

In this manner, the information processing device 100 alters the coordinates of an atom forming the target molecule for each alteration pattern 703 regarding the rotation angle and generates the coordinate data. This allows the information processing device 100 to specify a plurality of different states of the target molecule regarding the rotation angle. For example, the information processing device 100 may appropriately specify the coordinates of each atom of a plurality of atoms forming the target molecule in a plurality of different states of the target molecule.

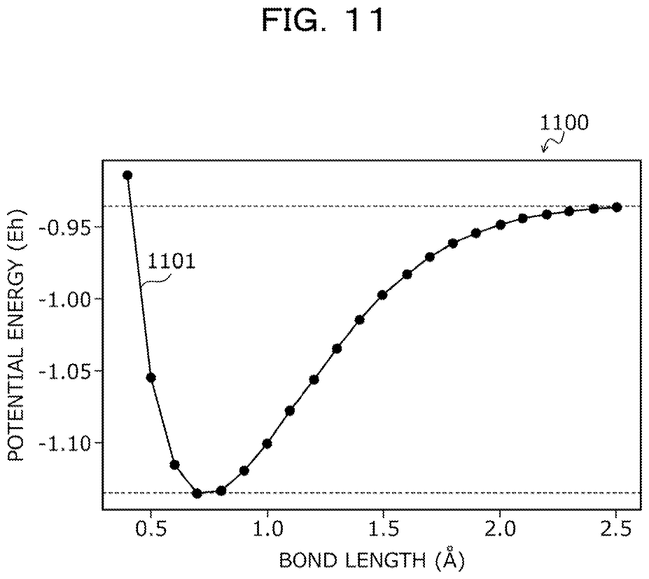

Next, an example in which the information processing device 100 generates the PEC will be described with reference to FIG. 11.

FIG. 11 is an explanatory diagram illustrating an example of generating the PEC. As illustrated in FIG. 11, the information processing device 100 calculates the potential energy of a certain target molecule for each piece of the coordinate data generated for the target molecule. The potential energy is, for example, Hartree energy or the like.

For example, the information processing device 100 calculates the potential energy of the target molecule, based on the coordinate data generated for each alteration pattern 701 regarding the bond length. For example, based on the calculated potential energy, the information processing device 100 generates a graph 1100 illustrating a PEC 1101 with the bond length on the abscissa axis and the potential energy on the ordinate axis. The information processing device 100 outputs the graph 1100 such that the user is allowed to refer to the graph 1100. This allows the information processing device 100 to make it possible for the user to refer to the PEC and to easily analyze the properties of the target molecule.

(First Acceptance Processing Procedure)

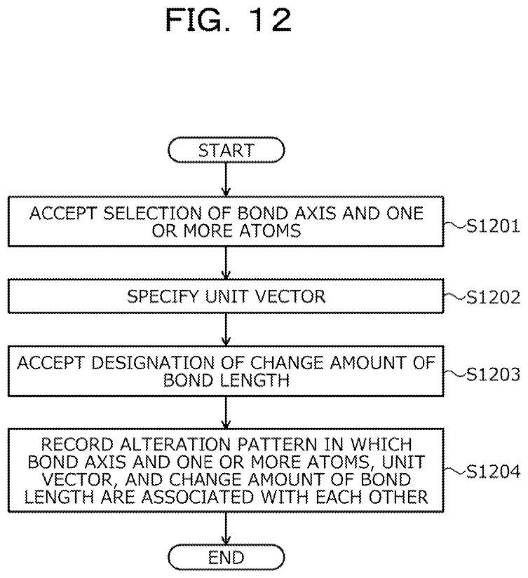

Next, an example of a first acceptance processing procedure executed by the information processing device 100 will be described with reference to FIG. 12. The first acceptance process is implemented by, for example, the CPU 301, a storage area such as the memory 302 or the recording medium 305, and the network I/F 303 illustrated in FIG. 3.

FIG. 12 is a flowchart illustrating an example of the first acceptance processing procedure. In FIG. 12, the information processing device 100 accepts selection of a bond axis whose bond length is to be changed and one or more atoms whose coordinates are to be altered, based on operation input from the user (step S1201). The one or more atoms include, for example, at least one atom coupled to the bond axis for which selection has been accepted.

Next, among two atoms coupled to the bond axis for which selection has been accepted, the information processing device 100 specifies the unit vector along the direction from the coordinates of one atom whose coordinates are to be altered, to another atom whose coordinates are not to be altered (step S1202). Then, the information processing device 100 accepts designation of the change amount of the bond length, based on operation input from the user (step S1203).

Next, the information processing device 100 records, in the pattern file, an alteration pattern in which the bond axis and the one or more atoms for which selection has been accepted, the specified unit vector, and the change amount of the bond length for which designation has been accepted are associated with each other (step S1204). Then, the information processing device 100 ends the first acceptance process. This allows the information processing device 100 to accept the alteration pattern and to make it possible to specify how to alter the coordinates of an atom forming the target molecule.

(Second Acceptance Processing Procedure)

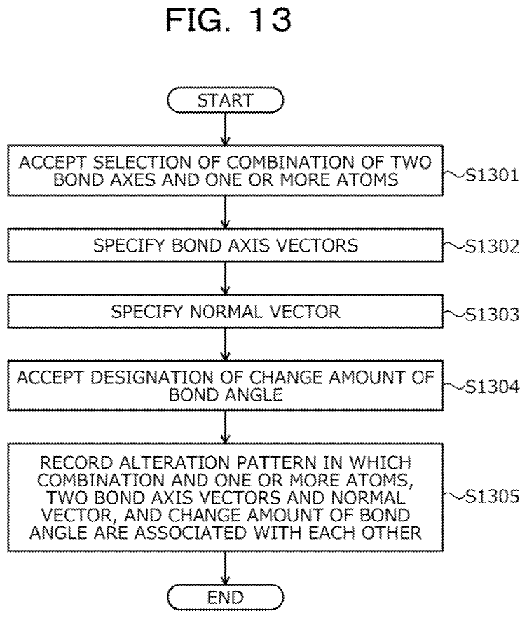

Next, an example of a second acceptance processing procedure executed by the information processing device 100 will be described with reference to FIG. 13. The second acceptance process is implemented by, for example, the CPU 301, a storage area such as the memory 302 or the recording medium 305, and the network I/F 303 illustrated in FIG. 3.

FIG. 13 is a flowchart illustrating an example of the second acceptance processing procedure. In FIG. 13, the information processing device 100 accepts selection of a combination of two bond axes whose bond angle is to be changed and one or more atoms whose coordinates are to be altered, based on operation input from the user (step S1301). The one or more atoms include, for example, at least one atom coupled to one bond axis of the combination for which selection has been accepted.

Next, the information processing device 100 specifies bond axis vectors from the coordinates of the atom at the center of the two bond axes (common to the two bond axes) of the combination for which selection has been accepted, to the coordinates of the other atom of each bond axis (step S1302). Then, the information processing device 100 specifies the normal vector with respect to the plane formed by the two bond axes of the combination for which selection has been accepted, based on the specified two bond axis vectors (step S1303). The information processing device 100 also accepts designation of the change amount of the bond angle, based on operation input from the user (step S1304).

Next, the information processing device 100 records, in the pattern file, an alteration pattern in which the combination and the one or more atoms for which selection has been accepted, the specified two bond axis vectors and normal vector, and the change amount of the bond angle for which designation has been accepted are associated with each other (step S1305). Then, the information processing device 100 ends the second acceptance process. This allows the information processing device 100 to accept the alteration pattern and to make it possible to specify how to alter the coordinates of an atom forming the target molecule.

(Third Acceptance Processing Procedure)

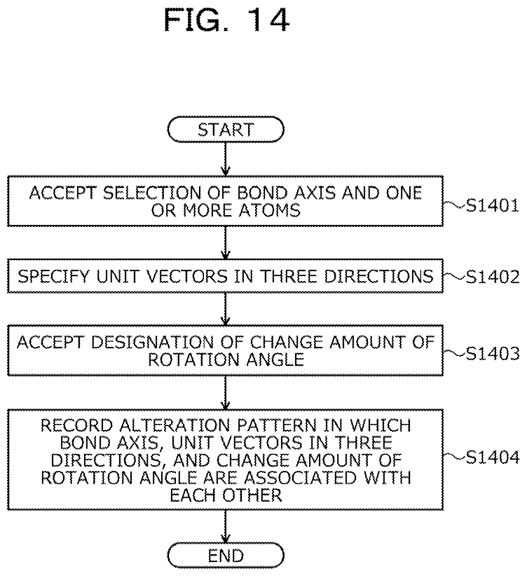

Next, an example of a third acceptance processing procedure executed by the information processing device 100 will be described with reference to FIG. 14. The third acceptance process is implemented by, for example, the CPU 301, a storage area such as the memory 302 or the recording medium 305, and the network I/F 303 illustrated in FIG. 3.

FIG. 14 is a flowchart illustrating an example of the third acceptance processing procedure. In FIG. 14, the information processing device 100 accepts selection of the bond axis to be assumed as the rotation axis whose rotation angle is to be changed and one or more atoms whose coordinates are to be altered, based on operation input from the user (step S1401). The one or more atoms include, for example, at least an atom coupled to one atom on the bond axis to be assumed as the rotation axis for which selection has been accepted.

Next, the information processing device 100 specifies unit vectors in three directions that are each horizontal or vertical to the bond axis for which selection has been accepted and are vertical to each other (step S1402). Then, the information processing device 100 accepts designation of the change amount of the rotation angle, based on operation input from the user (step S1403).

Next, the information processing device 100 records, in the pattern file, an alteration pattern in which the bond axis for which selection has been accepted, the specified unit vectors in the three directions, and the change amount of the rotation angle for which designation has been accepted are associated with each other (step S1404). Then, the information processing device 100 ends the third acceptance process. This allows the information processing device 100 to accept the alteration pattern and to make it possible to specify how to alter the coordinates of an atom forming the target molecule.

(Overall Processing Procedure)

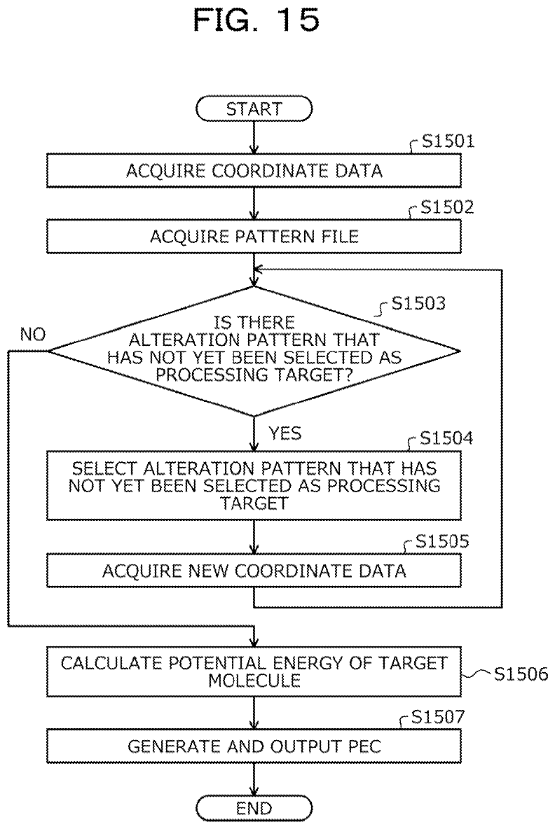

Next, an example of an overall processing procedure executed by the information processing device 100 will be described with reference to FIG. 15. The overall process is implemented by, for example, the CPU 301, a storage area such as the memory 302 or the recording medium 305, and the network I/F 303 illustrated in FIG. 3.

FIG. 15 is a flowchart illustrating an example of the overall processing procedure. In FIG. 15, the information processing device 100 acquires the coordinate data (step S1501). Next, the information processing device 100 acquires the pattern file (step S1502). Then, the information processing device 100 determines whether or not there is an alteration pattern that has not yet been selected as a processing target in the pattern file (step S1503).

Here, when all the alteration patterns have already been selected as processing targets (step S1503: No), the information processing device 100 proceeds to processing in step S1506. On the other hand, when there is an alteration pattern that has not yet been selected as a processing target (step S1503: Yes), the information processing device 100 proceeds to processing in step S1504.

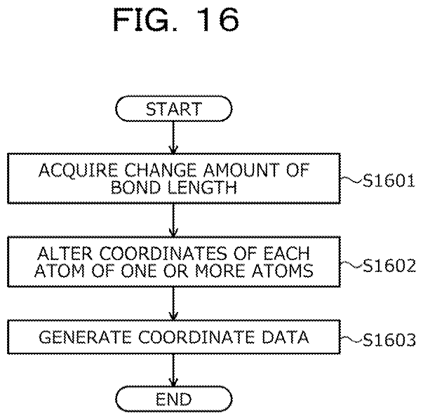

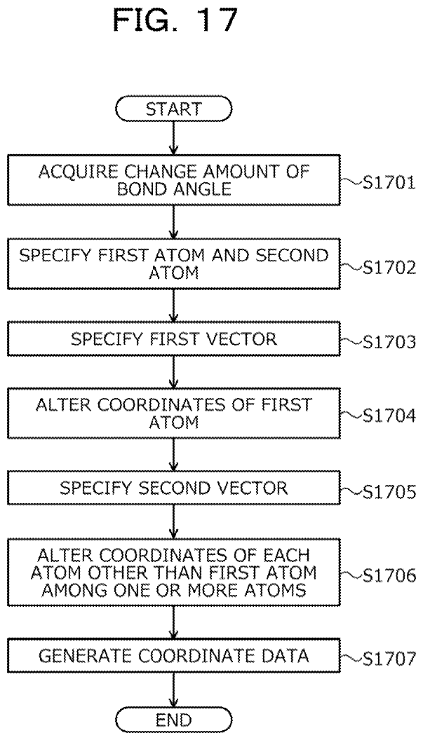

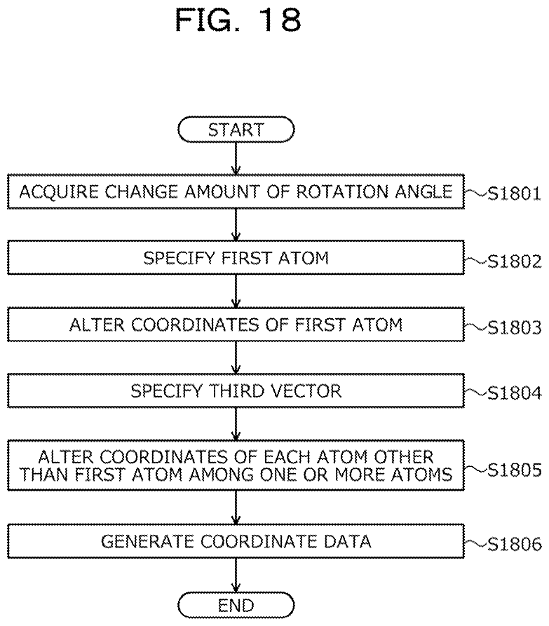

In step S1504, the information processing device 100 selects, as a processing target, an alteration pattern that has not yet been selected as a processing target from the pattern file (step S1504). Next, the information processing device 100 executes a first alteration process described later with reference to FIG. 16, a second alteration process described later with reference to FIG. 17, or a third alteration process described later with reference to FIG. 18 according to the selected alteration pattern and acquires new coordinate data (step S1505). Then, the information processing device 100 returns to processing in step S1503.

In step S1506, the information processing device 100 calculates the potential energy of the target molecule, based on each piece of the acquired coordinate data (step S1506). Next, the information processing device 100 generates and outputs the PEC, based on the calculated potential energy (step S1507). Then, the information processing device 100 ends the overall process. This allows the information processing device 100 to generate the PEC and to make it possible for the user to refer to the PEC and to easily analyze the properties of the target molecule.

(First Alteration Processing Procedure)

Next, an example of a first alteration processing procedure executed by the information processing device 100 will be described with reference to FIG. 16. The first alteration process is implemented by, for example, the CPU 301, a storage area such as the memory 302 or the recording medium 305, and the network I/F 303 illustrated in FIG. 3. Note that the processing in FIG. 16 is detailed processing of the processing in S1505 in FIG. 15 described above.

FIG. 16 is a flowchart illustrating an example of the first alteration processing procedure. In FIG. 16, the information processing device 100 acquires the change amount of the bond length, based on the alteration pattern (step S1601). Next, based on the alteration pattern, the information processing device 100 alters the coordinates of each atom of the one or more atoms whose selection has been accepted, to the coordinates at the destination after being moved by an amount equal to the unit vector multiplied by the change amount of the bond length (step S1602).

Then, the information processing device 100 generates coordinate data that indicates the coordinates of each atom of a plurality of atoms forming the target molecule, including the altered coordinates of each atom of the one or more atoms (step S1603). Thereafter, the information processing device 100 ends the first alteration process. This may allow the information processing device 100 to appropriately alter the coordinates of an atom forming the target molecule so as to make the coordinates correspond to the state obtained by changing the bond length.

(Second Alteration Processing Procedure)

Next, an example of a second alteration processing procedure executed by the information processing device 100 will be described with reference to FIG. 17. Note that FIG. 17 gives a description regarding detailed processing of the processing in S1505 in FIG. 15 described above. The second alteration process is implemented by, for example, the CPU 301, a storage area such as the memory 302 or the recording medium 305, and the network I/F 303 illustrated in FIG. 3.

FIG. 17 is a flowchart illustrating an example of the second alteration processing procedure. In FIG. 17, the information processing device 100 acquires the change amount of the bond angle, based on the alteration pattern (step S1701). Then, based on the alteration pattern, the information processing device 100 specifies, among the one or more atoms for which selection has been accepted, the first atom coupled to one of the bond axes of the combination for which selection has been accepted and the second atom at the center of the two bond axes of the combination (step S1702).

Next, the information processing device 100 specifies the first vector from the coordinates of the second atom to the coordinates of the first atom after the change in the bond angle, based on the change amount of the bond angle and the two bond axis vectors and the normal vector, based on the alteration pattern (step S1703). Then, the information processing device 100 alters the coordinates of the first atom to the coordinates at the destination after being moved from the coordinates by an amount equal to the specified first vector (step S1704).

Next, the information processing device 100 specifies the second vector from the coordinates of the first atom before the change in the bond angle to the coordinates of the first atom after the change in the bond angle (step S1705). Then, the information processing device 100 alters the coordinates of each atom other than the first atom among the one or more atoms for which selection has been accepted, to the coordinates at the destination after being moved from the coordinates by an amount equal to the specified second vector (step S1706).

Next, the information processing device 100 generates coordinate data indicating the coordinates of each atom of a plurality of atoms forming the target molecule, including the altered coordinates of each atom of the one or more atoms (step S1707). Then, the information processing device 100 ends the second alteration process. This may allow the information processing device 100 to appropriately alter the coordinates of an atom forming the target molecule so as to make the coordinates correspond to the state obtained by changing the bond angle.

(Third Alteration Processing Procedure)

Next, an example of a third alteration processing procedure executed by the information processing device 100 will be described with reference to FIG. 18. Note that the processing in FIG. 18 is detailed processing of the processing in S1505 in FIG. 15 described above. The third alteration process is implemented by, for example, the CPU 301, a storage area such as the memory 302 or the recording medium 305, and the network I/F 303 illustrated in FIG. 3.

FIG. 18 is a flowchart illustrating an example of the third alteration processing procedure. In FIG. 18, the information processing device 100 acquires the change amount of the rotation angle, based on the alteration pattern (step S1801).

Next, based on the alteration pattern, the information processing device 100 specifies, among the one or more atoms for which selection has been accepted, the first atom coupled to a rotating atom that is to be rotated and is coupled to the bond axis to be assumed as the rotation axis for which selection has been accepted (step S1802). Then, based on the alteration pattern, the information processing device 100 alters the coordinates of the first atom to coordinates obtained by multiplying the coordinates by a rotation matrix in which the change amount of the rotation angle is set (step S1803).

Next, the information processing device 100 specifies the third vector from the coordinates of the first atom before the change in the rotation angle to the coordinates of the first atom after the change in the rotation angle (step S1804). Then, the information processing device 100 alters the coordinates of each atom other than the first atom among the one or more atoms for which selection has been accepted, to the coordinates at the destination after being moved from the coordinates by an amount equal to the specified third vector (step S1805).

Next, the information processing device 100 generates coordinate data indicating the coordinates of each atom of a plurality of atoms forming the target molecule, including the altered coordinates of each atom of the one or more atoms (step S1806). Then, the information processing device 100 ends the third alteration process. This may allow the information processing device 100 to appropriately alter the coordinates of an atom forming the target molecule so as to make the coordinates correspond to the state obtained by changing the rotation angle.

As described above, according to the information processing device 100, selection of one or more atoms from among a plurality of atoms forming a target molecule existing in a predetermined coordinate space can be accepted. According to the information processing device 100, at least one of the unit vector, the normal vector, or the rotation matrix in the coordinate space can be specified according to designation of the change amount of at least one of the bond length, the bond angle, or the rotation angle. According to the information processing device 100, the coordinates of each atom of the one or more atoms in the coordinate space can be altered based on at least one of the specified unit vector, normal vector, or rotation matrix in the coordinate space. This may allow the information processing device 100 to appropriately alter the coordinates of each atom of the one or more atoms and to facilitate to specify a new state of the target molecule. The information processing device 100 may reduce the workload put on the user when specifying a new state of the target molecule.

According to the information processing device 100, the coordinates of each atom of the one or more atoms can be altered to the coordinates at the destination after being moved from the coordinates by an amount equal to the unit vector multiplied by the first change amount of the bond length, according to designation of the first change amount. This may allow the information processing device 100 to appropriately alter the coordinates of each atom of the one or more atoms so as to make the coordinates correspond to a case where the bond length is changed.

According to the information processing device 100, the first vector from the coordinates of the second atom to the coordinates of the first atom after the change in the bond angle can be specified based on the second change amount of the bond angle, the vector from the coordinates of the second atom to the coordinates of the third atom, and the normal vector. According to the information processing device 100, the coordinates of the first atom can be altered to the coordinates at the destination after being moved from the coordinates by an amount equal to the specified first vector. According to the information processing device 100, if the one or more atoms include an atom other than the first atom, the second vector between the coordinates of the first atom before and after the change in the bond angle can be specified. According to the information processing device 100, the coordinates of each atom other than the first atom among the one or more atoms can be altered to the coordinates at the destination after being moved from the coordinates by an amount equal to the specified second vector. This may allow the information processing device 100 to appropriately alter the coordinates of each atom of the one or more atoms so as to make the coordinates correspond to a case where the bond angle is changed.

According to the information processing device 100, the coordinates of the first atom can be altered to coordinates obtained by multiplying the coordinates by the rotation matrix corresponding to the third change amount of the rotation angle, according to designation of the third change amount. According to the information processing device 100, if the one or more atoms include an atom other than the first atom, the third vector between the coordinates of the first atom before and after the change in the rotation angle can be specified. According to the information processing device 100, the coordinates of each atom other than the first atom among the one or more atoms can be altered to the coordinates at the destination after being moved from the coordinates by an amount equal to the specified third vector. This may allow the information processing device 100 to appropriately alter the coordinates of each atom of the one or more atoms so as to make the coordinates correspond to a case where the rotation angle is changed.

According to the information processing device 100, the coordinates of each atom of the one or more atoms in the coordinate space can be altered for each designation of the change amount of at least one of the bond length, the bond angle, or the rotation angle. According to the information processing device 100, the potential energy of the target molecule in a case where the coordinates of each atom of the one or more atoms in the coordinate space are altered according to the change amount for each designation of the change amount can be calculated. According to the information processing device 100, the PEC of the target molecule can be generated based on the calculated potential energy. This allows the information processing device 100 to make it possible for the user to refer to the PEC and to easily analyze the properties of the target molecule.

According to the information processing device 100, by accepting selection of the first atom and selecting another atom linked with the first atom from among a plurality of atoms, selection of the one or more atoms including the first atom and the another atom can be accepted. This may allow the information processing device 100 to reduce the workload put on the user when selecting one or more atoms.

Note that the information processing method described in the present embodiment can be implemented by executing a program prepared in advance on a computer such as a PC or a workstation. The information processing program described in the present embodiment is executed by being recorded on a computer-readable recording medium and being read from the recording medium by the computer. The recording medium is a hard disk, a flexible disk, a compact disc (CD)-ROM, a magneto-optical disc (MO), a digital versatile disc (DVD), or the like.

In addition, the information processing program described in the present embodiment may be distributed via a network such as the Internet.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing an information processing program for causing a computer to execute a process comprising:
accepting selection of one or more atoms among a plurality of atoms that form a target molecule that exists in a predetermined coordinate space; and
altering coordinates of each atom of the one or more atoms from first coordinates to second coordinates in the coordinate space, according to designation of a change amount of: a bond length between a first atom included in the one or more atoms for which the selection has been accepted and a second atom that is not included in the one or more atoms but is coupled to the first atom; a bond angle formed by a first bond axis that bonds the first atom and the second atom and a second bond axis that bonds the second atom and a third atom that is not included in the one or more atoms but is coupled to the second atom; or a rotation angle of the second atom with respect to the second bond axis; or any combination of the bond length, the bond angle, or the rotation angle, based on: a unit vector between the coordinates of the first atom and the second atom of which the bond length is to be changed; a normal vector with respect to a plane formed by the first bond axis and the second bond axis of which the bond angle is to be changed; or a rotation matrix of the second atom of which the rotation angle is to be changed; or any combination of the unit vector, the normal vector, or the rotation matrix, in the coordinate space.

2. The non-transitory computer-readable recording medium according to claim 1, wherein the altering includes
altering the first coordinates of each atom of the one or more atoms to the second coordinates that are a destination after being moved from the first coordinates of each atom by an amount equal to the unit vector multiplied by a first change amount of the bond length, according to the designation of the first change amount.

3. The non-transitory computer-readable recording medium according to claim 1, wherein the altering includes:
specifying a first vector from the coordinates of the second atom to the second coordinates of the first atom after the change in the bond angle, based on a second change amount of the bond angle, a vector from the coordinates of the second atom to the coordinates of the third atom, and the normal vector, according to the designation of the second change amount; altering the first coordinates of the first atom to the second coordinates at a destination after being moved from the first coordinates of the first atom by an amount equal to the specified first vector; specifying a second vector from the first coordinates to the second coordinates of the first atom before and after the change in the bond angle, when the one or more atoms include the atoms other than the first atom; and altering the first coordinates of each atom other than the first atom among the one or more atoms to the second coordinates at the destination after being moved from the first coordinates of each atom by the amount equal to the specified second vector.

4. The non-transitory computer-readable recording medium according to claim 1, wherein the altering includes:
altering the first coordinates of the first atom to the second coordinates obtained by multiplying the first coordinates of the first atom by the rotation matrix that corresponds to a third change amount of the rotation angle amount, according to the designation of the third change amount; specifying a third vector from the first coordinates to the second coordinates of the first atom before and after the change in the rotation angle, when the one or more atoms include the atoms other than the first atom; and altering the first coordinates of each atom other than the first atom among the one or more atoms to the second coordinates at a destination after being moved from the first coordinates of each atom by an amount equal to the specified third vector.

5. The non-transitory computer-readable recording medium according to claim 1, for causing the computer to execute the process comprising:
calculating potential energy of the target molecule separately for each form of the target molecule after the alteration in which the coordinates of each atom of the one or more atoms are altered from the first coordinates to the second coordinates in the target molecule, according to each case of the designation of the change amount; and
generating a potential energy curve of the target molecule, based on a plurality of calculated results of the potential energy.

6. The non-transitory computer-readable recording medium according to claim 1, wherein the accepting the selection includes:
accepting the selection of the first atom; and accepting the selection of the one or more atoms that include the first atom and another atom linked with the first atom, by selecting the another atom from among the plurality of atoms.

7. An information processing method comprising:
accepting selection of one or more atoms among a plurality of atoms that form a target molecule that exists in a predetermined coordinate space; and
altering coordinates of each atom of the one or more atoms from first coordinates to second coordinates in the coordinate space, according to designation of a change amount of: a bond length between a first atom included in the one or more atoms for which the selection has been accepted and a second atom that is not included in the one or more atoms but is coupled to the first atom; a bond angle formed by a first bond axis that bonds the first atom and the second atom and a second bond axis that bonds the second atom and a third atom that is not included in the one or more atoms but is coupled to the second atom; or a rotation angle of the second atom with respect to the second bond axis; or any combination of the bond length, the bond angle, or the rotation angle, based on: a unit vector between the coordinates of the first atom and the second atom of which the bond length is to be changed; a normal vector with respect to a plane formed by the first bond axis and the second bond axis of which the bond angle is to be changed; or a rotation matrix of the second atom of which the rotation angle is to be changed; or any combination of the unit vector, the normal vector, or the rotation matrix, in the coordinate space.

8. An information processing device comprising:
a memory; and
a processor coupled to the memory and configured to:
accept selection of one or more atoms among a plurality of atoms that form a target molecule that exists in a predetermined coordinate space; and
alter coordinates of each atom of the one or more atoms from first coordinates to second coordinates in the coordinate space, according to designation of a change amount of: a bond length between a first atom included in the one or more atoms for which the selection has been accepted and a second atom that is not included in the one or more atoms but is coupled to the first atom; a bond angle formed by a first bond axis that bonds the first atom and the second atom and a second bond axis that bonds the second atom and a third atom that is not included in the one or more atoms but is coupled to the second atom; or a rotation angle of the second atom with respect to the second bond axis; or any combination of the bond length, the bond angle, or the rotation angle, based on: a unit vector between the coordinates of the first atom and the second atom of which the bond length is to be changed; a normal vector with respect to a plane formed by the first bond axis and the second bond axis of which the bond angle is to be changed; or a rotation matrix of the second atom of which the rotation angle is to be changed; or any combination of the unit vector, the normal vector, or the rotation matrix, in the coordinate space.
